(19) United States
(12) Patent Application Publication
(10) Pub. No.: US 2024/0024328 A1
(43) Pub. Date: Jan. 25, 2024

(54) RUXOLITINIB FOR THE TREATMENT OF PRURIGO NODULARIS

(71) Applicant: Incyte Corporation, Wilmington, DE (US)

(72) Inventors: Paul Smith, Wilmington, DE (US); Jörg Wenzel, Bonn (DE)

(21) Appl. No.: 18/480,898

(22) Filed: Oct. 4, 2023

Related U.S. Application Data
(63) Continuation of application No. 17/735,273, filed on May 3, 2022.
(60) Provisional application No. 63/183,225, filed on May 3, 2021.

(51) Int. Cl. A61K 31/519; A61P 17/00; A61K 9/00; A61K 9/06; A61K 45/06

(52) U.S. Cl. CPC A61K 31/519; A61P 17/00; A61K 9/0014; A61K 9/06; A61K 45/06

(57) ABSTRACT

This disclosure relates to ruxolitinib, or a pharmaceutically acceptable salt thereof, and its use in treating prurigo nodularis.

The present application claims the benefit of U.S. Provisional Application No. 63/183,225, filed May 3, 2021, which is incorporated herein by reference in its entirety.

### TECHNICAL FIELD

This disclosure relates to ruxolitinib, or a pharmaceutically acceptable salt thereof, for use thereof in treating prurigo nodularis.

### BACKGROUND

Prurigo nodularis (PN) is a chronic skin disorder characterized by firm, dome-shaped, intensely pruritic nodules ranging in size from a few millimeters to several centimeters. The nodules are often symmetrically distributed on the extensor surfaces of the arms and legs and on the trunk. PN has an estimated prevalence of 72 per 100,000 persons in the US, based on people with health insurance. Reports vary on whether it more frequently in women versus men or occurs equally in men and women. People with darker or greater degrees of pigmentation in their skin are substantially more likely to have PN than patients with lighter or less pigmentation in their skin. In particular, one study found that African American patients were 3.4 times more likely to have PN than Caucasian patients.

Quality of life can be substantially negatively impacted by PN. In particular, quality of life issues include sleep disturbance, impact on job performance, and avoidance of social activities. In addition, the burden of systemic comorbidities in prurigo nodularis often exceeds that of other inflammatory skin disorders (e.g., atopic dermatitis or psoriasis). Prurigo nodularis is associated with increased rates of mental health (specifically anxiety and depression), endocrine, cardiovascular, and renal disorders, as well as HIV and malignancy. Approximately half of all patients with PN report a history of atopic dermatitis.

Pharmacologic therapy with first-generation sedating antihistamines (e.g., hydroxyzine, diphenhydramine) administered at bedtime may be useful in controlling nocturnal pruritus. Both selective serotonin reuptake inhibitors and tricyclic antidepressants are also employed for chronic pruritus, especially when a component of depression is present.

Super-potent topical corticosteroids are considered first-line therapy. Patients with widespread disease may be given phototherapy. Patients with recalcitrant PN may be given systemic treatments including systemic immunosuppressants, thalidomide, lenalidomide, and anticonvulsants. These treatments are associated with potential significant toxicity, and their efficacy in patients with recalcitrant PN has not been established. Accordingly, there is a need to develop new therapies for the treatment of prurigo nodularis. This application addresses this need and others.

### DESCRIPTION OF THE DRAWINGS

FIG. 1 depicts a representation of skin punch biopsies used for the JAK1/JAK2 mediated pharmacological inhibition of PN pathophysiology.

FIG. 2 depicts a graphical representation of JAK1/JAK2 mediated pharmacological inhibition of PN pathophysiology using JAK1/JAK2 inhibitor ruxolitinib.

### SUMMARY

Provided herein are methods for the treatment of prurigo nodularis in a subject in need thereof, comprising administering to the subject a therapeutically effective amount of ruxolitinib, or a pharmaceutically acceptable salt thereof.

Provided herein is ruxolitinib, or a pharmaceutically acceptable salt thereof, for the treatment of prurigo nodularis in a subject in need thereof.

Provided herein is a use of ruxolitinib, or a pharmaceutically acceptable salt thereof, for manufacture of a medicament for use in treating prurigo nodularis in a subject in need thereof.

### DETAILED DESCRIPTION

The present invention provides, inter alia, a method of treating prurigo nodularis in a subject in need thereof, comprising administering to the subject a therapeutically effective amount of ruxolitinib, or a pharmaceutically acceptable salt thereof. Ruxolitinib is a JAK1/JAK2 inhibitor which is selective for JAK1 and JAK2 over JAK3.

In some embodiments, the ruxolitinib is present in the form of ruxolitinib phosphate.

In some embodiments, the ruxolitinib is present in the form of ruxolitinib phosphate (1:1 salt).

In some embodiments, the ruxolitinib, or a pharmaceutically acceptable salt thereof, is administered topically to an affected skin area.

In some embodiments, the ruxolitinib, or a pharmaceutically acceptable salt thereof, is an administered as a topical formulation.

In some embodiments, the topical formulation is an administered as a topical formulation comprising about 0.1% to about 3% of the ruxolitinib, or the pharmaceutically acceptable salt thereof, on a free base basis.

In some embodiments, the topical formulation is an administered as a topical formulation comprising about 0.5% to about 2% of the ruxolitinib, or the pharmaceutically acceptable salt thereof, on a free base basis.

In some embodiments, the topical formulation is an administered as a topical formulation comprising about 0.5% to about 1.5% of the ruxolitinib, or the pharmaceutically acceptable salt thereof, on a free base basis.

In some embodiments, the topical formulation is an administered as a topical formulation comprising about 0.75% to about 1.5% of the ruxolitinib, or the pharmaceutically acceptable salt thereof, on a free base basis.

In some embodiments, the topical formulation is an administered as a topical formulation comprising about 0.5% of the ruxolitinib, or the pharmaceutically acceptable salt thereof, on a free base basis. In some embodiments, the topical formulation is an administered as a topical formulation comprising about 0.75% of the ruxolitinib, or the pharmaceutically acceptable salt thereof, on a free base basis. In some embodiments, the topical formulation is an administered as a topical formulation comprising about 1% of the ruxolitinib, or the pharmaceutically acceptable salt thereof, on a free base basis. In some embodiments, the topical formulation is an administered as a topical formulation comprising about 1.5% of the ruxolitinib, or the pharmaceutically acceptable salt thereof, on a free base basis.

In some embodiments, the topical formulation is administered twice per day (BID). In some embodiments, the topical formulation is administered once per day (QD).

In some embodiments, the administering is continued for 12 weeks.

In some embodiments, the administering is continued every day for 12 weeks.

In some embodiments, the ruxolitinib, or pharmaceutically acceptable salt thereof, is administered in combination with a further therapeutic agent.

In some embodiments, the administering comprises administering the ruxolitinib, or pharmaceutically acceptable salt thereof, together with at least one pharmaceutically acceptable carrier or excipient.

In some embodiments, efficacy of the treatment method disclosed herein can be established based upon an Investigator's Global Assessment (IGA). In some embodiments, the IGA-TS (Investigator's Global Assessment Treatment Success) is defined as an IGA score of 0 or 1 with ≥2 grade improvement from baseline. Efficacy may be established if by evaluating a proportion of subjects achieving an IGA-TS (IGA of 0 or 1 with a 2-point decrease) at a designated point of time (e.g., week 2, week 4, week 8, or week 16).

In some embodiments, efficacy of the treatment method disclosed herein can be established based upon Itch Numerical Rating Scale (Itch NRS). In some embodiments, efficacy may be demonstrated by achieving a preset proportion of subjects achieving at least 2 or 4-point improvement in Itch NRS (e.g., at Week 16). In some embodiments, efficacy may be demonstrated by observing time to ≥2-point or ≥4-point improvement from baseline in Itch NRS. In some embodiments, the ruxolitinib, or the pharmaceutically acceptable salt thereof, and/or methods of use described herein result in an improvement in a subject's response to Itch NRS from baseline. In some embodiments, the ruxolitinib, or the pharmaceutically acceptable salt thereof, and/or methods of use described herein result in about a 5%, about a 10%, about a 20%, about a 30%, about a 40%, about a 50%, about a 60%, about a 70%, about a 80%, about a 90%, or about a 95% improvement in a subject's response to Itch NRS from baseline. The itch NRS is a daily patient-reported measure (24-hour recall) of itch intensity. Subjects will be asked to rate the itching severity because of their AD by selecting a number from 0 (no itch) to 10 (worst imaginable itch) that best describes their worst level of itching in the past 24 hours. In a non-limiting example, patients can be issued a hand-held device (eDiary) on which to record itch severity. The patient can be instructed to complete the eDiary each night.

In some embodiments, efficacy of the treatment method disclosed herein can be established based upon the Prurigo Activity Score (PAS). In some embodiments, the ruxolitinib, or the pharmaceutically acceptable salt thereof, and/or methods of use described herein result in an improvement in a subject's response in Prurigo Activity Score (PAS) from baseline. In some embodiments, the ruxolitinib, or the pharmaceutically acceptable salt thereof, and/or methods of use described herein result in about a 5%, about a 10%, about a 20%, about a 30%, about a 40%, about a 50%, about a 60%, about a 70%, about a 80%, about a 90%, or about a 95% improvement in a subject's response in Prurigo Activity Score (PAS) from baseline. The activity in terms of percentage of PN lesions with excoriations/crusts on top (reflecting active scratching) and the percentage of healed pruriginous lesions is measured by the subitem “Activity” of PAS in order to quantify change of PN skin lesions.

In some embodiments, efficacy of the treatment method disclosed herein can be established based upon Itchy Quality of Life (Itchy QoL). In some embodiments, the ruxolitinib, or the pharmaceutically acceptable salt thereof, and/or methods of use described herein result in an improvement in a subject's response in Itchy Quality of Life (Itchy QoL) from baseline. In some embodiments, the ruxolitinib, or the pharmaceutically acceptable salt thereof, and/or methods of use described herein result in about a 5%, about a 10%, about a 20%, about a 30%, about a 40%, about a 50%, about a 60%, about a 70%, about a 80%, about a 90%, or about a 95% improvement in a subject's response in Itchy Quality of Life (Itchy QoL) from baseline. ItchyQoL is a pruritus-specific instrument that was developed and validated by Chen et al., Arch Dermatol, 147(10):1153-6 (2011). The 22-question questionnaire can be applied to patients with pruritus, independent of the underlying cause. ItchyQoL is a 3-dimensional instrument, which contains the domains symptoms, functioning and emotions.

In some embodiments, efficacy of the treatment method disclosed herein can be established based upon Dynamic Pruritus Score (DPS). In some embodiments, the ruxolitinib, or the pharmaceutically acceptable salt thereof, and/or methods of use described herein result in an improvement in a subject's response in Dynamic Pruritus Score (DPS) from baseline. In some embodiments, the ruxolitinib, or the pharmaceutically acceptable salt thereof, and/or methods of use described herein result in about a 5%, about a 10%, about a 20%, about a 30%, about a 40%, about a 50%, about a 60%, about a 70%, about a 80%, about a 90%, or about a 95% improvement in a subject's response in Dynamic Pruritus Score (DPS) from baseline. The DPS evaluates the change in pruritus intensity, compared with a defined earlier time-point, and is comprised of a horizontal line measuring itch improvement via 11 marks on a scale, each labelled with both a numerical and verbal description.

In some embodiments, efficacy of the treatment method disclosed herein can be established based upon patient-reported outcomes (PROs). In some embodiments, efficacy of the treatment method disclosed herein can be established based upon a Dermatology Life Quality Index (DLQI). In some embodiments, the ruxolitinib, or the pharmaceutically acceptable salt thereof, and/or methods of use described herein result in an improvement in a subject's response to DLQI from baseline. In some embodiments, the ruxolitinib, or the pharmaceutically acceptable salt thereof, and/or methods of use described herein result in about a 5%, about a 10%, about a 20%, about a 30%, about a 40%, about a 50%, about a 60%, about a 70%, about a 80%, about a 90%, or about a 95% improvement in a subject's response to DLQI from baseline. A dermatology-specific Quality of Life instrument. It is a validated 10-question questionnaire that has been used in over 40 different skin conditions in over 80 countries and is available in over 90 languages.

In some embodiments, efficacy can be evaluated based upon improvement in a subject's PROMIS Sleep Scale. In some embodiments, the ruxolitinib, or pharmaceutically acceptable salt thereof, and/or methods of use described herein result in an improvement in a subject's response to PROMIS Sleep Scale from baseline. In some embodiments, the ruxolitinib, or the pharmaceutically acceptable salt thereof, and/or methods of use described herein result in about a 5%, about a 10%, about a 20%, about a 30%, about a 40%, about a 50%, about a 60%, about a 70%, about a 80%, about a 90%, or about a 95% improvement in a subject's response to PROMIS Sleep Scale from baseline.

In some embodiments, the patient has an itch Numerical Rating Scale of at least 4 at baseline.

In some embodiments, the patient is aged 18 or older.

The term “about” means “approximately” (e.g., plus or minus approximately 10% of the indicated value).

In some embodiments, the ruxolitinib, or the pharmaceutically acceptable salt thereof, is administered as one or more sustained release dosage forms each comprising the ruxolitinib, or the pharmaceutically acceptable salt thereof.

The embodiments described herein are intended to be combined in any suitable combination as if the embodiments are multiply dependent claims (e.g., the embodiments related to ruxolitinib and doses of the same, the embodiments related to any salt forms of the compounds disclosed herein, the embodiments related to the individual types of cytokine related diseases or disorders, and the embodiments related to composition and/or administration can be combined in any combination).

All possible combinations are not separately listed herein merely for the sake of brevity.

Compounds described herein can also include isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to 2 H (also written as D for deuterium), ³H (also written as T for tritium), ¹¹C, ¹³C, ¹⁴C, ¹³N, ¹⁵N, ¹O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formulae (I), (II), or (III) can be optionally substituted with deuterium atoms, such as —CD₃ being substituted for —CH₃).The term, “compound,” as used herein is meant to include all stereoisomers, geometric isomers, tautomers, and isotopes of the structures depicted, unless the name indicates a specific stereoisomer. Compounds herein identified by name or structure as one particular tautomeric form are intended to include other tautomeric forms unless otherwise specified.

All compounds, and pharmaceutically acceptable salts thereof, can be found together with other substances such as water and solvents (e.g. hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “subject”, “individual,” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the ruxolitinib, or the pharmaceutically acceptable salt thereof, is administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the ruxolitinib, or the pharmaceutically acceptable salt thereof, can prevent prurigo nodularis in an individual who may be predisposed to the disease. The term “preventing” refers to blocking the occurrence of disease in a patient who may be predisposed to the disease but does not yet experience or display the pathology or symptomatology of the disease.

### Combination Therapies

The methods described herein can further comprise administering one or more additional therapeutic agents. The one or more additional therapeutic agents can be administered to a patient simultaneously or sequentially. The one or more additional therapeutic agents can be administered using different methods (e.g., topically).

In some embodiments, the additional therapeutic agent is selected from JAK inhibitors. Additional JAK inhibitors may include ATI-50002 (JAK1/3 selective). Additional JAK inhibitors may include PF-06651600 (JAK3 selective). Additional JAK inhibitors may include PF06700841 (JAK1/TYK2 selective). Additional JAK inhibitors may include baricitinib (JAK1/JAK2 selective). Additional JAK inhibitors may include TYK2 selective inhibitors.

In some embodiments, the additional therapeutic agent is selected from antioxidants. Antioxidants may be selected from pseudocatalase, vitamin E, vitamin C, ubiquinone, lipoic acid, Polypodium leucotomos, catalase/superoxide dismutase combination, and Ginkgo biloba. In some embodiments, antioxidants may be further administered in combination with phototherapy. The administration of antioxidants during or before phototherapy aims to counteract the oxidative stress induced by UV radiation itself, increasing the phototherapy effectiveness.

In some embodiments, the additional therapeutic agent includes anti-histamines.

In some embodiments, the additional therapeutic agent is an antimetabolite. Antimetabolites may include 5-fluorouracil.

In some embodiments, the additional therapeutic agent is selected from topical corticosteroids, immunomodulators, calcineurin inhibitors, and phototherapy. In some embodiments, the additional therapies are systemic steroids or immunosuppressants.

In some embodiments, the additional therapeutic agent includes steroids (e.g., orally administered steroids) including systemic steroids. Steroid treatment may include oral steroid minipulse therapy (e.g., using betamethasone and/or dexamethasone).

In some embodiments, topical corticosteroids are selected from augmented betamethasone dipropionate, clobetasol propionate, diflorasone diacetate, halobetasol propionate amcinonide, betamethasone valerate, desoximetasone, diflorasone diacetate, fluocinolone acetonide, halcinonide, and triamcinolone acetonide.

In some embodiments, the additional therapeutic agent includes immunomodulators. Immunomodulators may include anti-IL15 therapy (e.g., AMG 714 monoclonal antibody). Immunomodulators may include anti-IL36 therapy (e.g., imsidolimab and spesolimab). Immunomodulators may include anti-TNFalpha therapy (e.g., etanercept and infliximab).

In some embodiments, immunomodulators are selected from apremilast, crisaborole, afamelanotide, minocycline, zinc, tofacitinib, AMG 714 monoclonal antibodies, imsidolimab, spesolimabcyclosporine, etanercept, infliximab, cyclophosphamide, ciclosporin, methotrexate, and sodium oxo-dihydro-acridinylacetate (ODHAA).

In some embodiments, calcineurin inhibitors are selected from tacrolimus (FK-506) and pimecrolimus.

In some embodiments, phototherapy includes exposure to ultraviolet (e.g., excimer lamps or lasers).

In some embodiments, the additional therapeutic agent is a Janus kinase inhibitor. In some embodiments, the Janus kinase inhibitor is administered topically.

In some embodiments, the additional therapeutic agent is a Neurokinin 1 receptor antagonists (e.g., Aprepitant).

In some embodiments, the additional therapeutic agent include anti-IL-4/IL-13 antibodies. In some embodiments, anti-IL-4/IL-13 antibodies are selected from dupilumab, lebrikizumab and tralokinumab.

In some embodiments, the additional therapeutic agent include anti-IL-5 antibodies. In some embodiments, anti-IL-5 antibodies are selected from benralizumab, mepolizumab, and reslizumab.

In some embodiments, the additional therapeutic agent include anti-IL-31 antibodies. In some embodiments, anti-IL-31 antibodies include nemolizumab.

In some embodiments, the additional therapeutic agent is an IL-6 antagonist or receptor antagonist. In some embodiments, the IL-6 receptor antagonist is tocilizumab.

### Pharmaceutical Formulations and Dosage Forms

When employed as pharmaceuticals, the ruxolitinib, or the pharmaceutically acceptable salt thereof, can be administered in the form of a pharmaceutical composition. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, foams, liquids and powders. Conventional pharmaceutical carriers, aqueous, powder or oily bases, thickeners and the like may be necessary or desirable.

This invention also includes pharmaceutical compositions which contain, as the active ingredient, the ruxolitinib, or the pharmaceutically acceptable salt thereof, described herein, or a pharmaceutically acceptable salt thereof, in combination with one or more pharmaceutically acceptable carriers (excipients). In some embodiments, the composition is suitable for topical administration. In making the compositions, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The ruxolitinib, or the pharmaceutically acceptable salt thereof, may be milled using known milling procedures such as wet milling to obtain a particle size appropriate for tablet formation and for other formulation types. Finely divided (nanoparticulate) preparations can be prepared by processes known in the art, e.g., see International App. No. WO 2002/000196.

The compositions can be formulated in a unit dosage form, each dosage containing a set amount of the active ingredient as the free form or a salt form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

Similar dosages may be used of the compounds described herein in the methods and uses of the invention.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present invention can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents such as a chemotherapeutic, steroid, anti-inflammatory compound, or immunosuppressant, examples of which are listed herein.

In some embodiments, the topical formulation is a cream formulation. In some embodiments, the cream formulation is an oil-in-water emulsion. In some embodiments, the cream is a solubilized cream. In some embodiments, the cream has a pH from about 2.8 to about 3.6. In the context of pH, “about” refers to ±0.3 (preferably ±0.2 or more preferably ±0.1).

In some embodiments, the cream comprises an oil-in-water emulsion, comprising 1.5% (w/w) on a free base basis of ruxolitinib phosphate.

In some embodiments, the cream is an oil-in-water emulsion as described in US 2015/0250790, which is incorporated herein by reference in its entirety. In particular, Examples 3-6 of US 2015/0250790 (and particularly Tables 3-5 and accompanying text) are incorporated herein by reference.

In some embodiments, the oil component is present in an amount of about 10% to about 40% by weight of the emulsion.

In some embodiments, the oil component is present in an amount of about 10% to about 24% by weight of the emulsion.

In some embodiments, the oil component is present in an amount of about 15% to about 24% by weight of the emulsion.

In some embodiments, the oil component is present in an amount of about 18% to about 24% by weight of the emulsion.

In some embodiments, the oil component comprises one or more substances independently selected from petrolatums, fatty alcohols, mineral oils, triglycerides, and silicone oils.

In some embodiments, the oil component comprises one or more substances independently selected from white petrolatum, cetyl alcohol, stearyl alcohol, light mineral oil, medium chain triglycerides, and dimethicone.

In some embodiments, the oil component comprises an occlusive agent component.

In some embodiments, the occlusive agent component is present in an amount of about 2% to about 15% by weight of the emulsion.

In some embodiments, the occlusive agent component is present in an amount of about 5% to about 10% by weight of the emulsion.

In some embodiments, the occlusive agent component comprises one or more substances selected from fatty acids (e.g., lanolin acid), fatty alcohols (e.g., lanolin alcohol), hydrocarbon oils & waxes (e.g., petrolatum), polyhydric alcohols (e.g., propylene glycol), silicones (e.g., dimethicone), sterols (e.g., cholesterol), vegetable or animal fat (e.g., cocoa butter), vegetable wax (e.g., Carnauba wax), and wax ester (e.g., bees wax).

In some embodiments, the occlusive agent component comprises one or more substances selected from lanolin acid fatty alcohols, lanolin alcohol, petrolatum, propylene glycol, dimethicone, cholesterol, cocoa butter, Carnauba wax, and bees wax.

In some embodiments, the occlusive agent component comprises petrolatum.

In some embodiments, the occlusive agent component comprises white petrolatum.

In some embodiments, the oil component comprises a stiffening agent component.

In some embodiments, the stiffening agent component is present in an amount of about 2% to about 8% by weight of the emulsion.

In some embodiments, the stiffening agent component is present in an amount of about 3% to about 6% by weight of the emulsion.

In some embodiments, the stiffening agent component is present in an amount of about 4% to about 7% by weight of the emulsion.

In some embodiments, the stiffening agent component comprises one or more substances independently selected from fatty alcohols.

In some embodiments, the stiffening agent component comprises one or more substances independently selected from C₁₂₋₂₀ fatty alcohols.

In some embodiments, the stiffening agent component comprises one or more substances independently selected from C₁₆₋₁₈ fatty alcohols.

In some embodiments, the stiffening agent component comprises one or more substances independently selected from cetyl alcohol and stearyl alcohol.

In some embodiments, the oil component comprises an emollient component.

In some embodiments, the emollient component is present in an amount of about 5% to about 15% by weight of the emulsion.

In some embodiments, the emollient component is present in an amount of about 7% to about 13% by weight of the emulsion.

In some embodiments, the emollient component comprises one or more substances independently selected from mineral oils and triglycerides.

In some embodiments, the emollient component comprises one or more substances independently selected from light mineral oil and medium chain triglycerides.

In some embodiments, the emollient component comprises one or more substances independently selected from light mineral oil, medium chain triglycerides, and dimethicone.

In some embodiments, the water is present in an amount of about 35% to about 65% by weight of the emulsion.

In some embodiments, the water is present in an amount of about 40% to about 60% by weight of the emulsion.

In some embodiments, the water is present in an amount of about 45% to about 55% by weight of the emulsion.

In some embodiments, the emulsifier component is present in an amount of about 1% to about 9% by weight of the emulsion.

In some embodiments, the emulsifier component is present in an amount of about 2% to about 6% by weight of the emulsion.

In some embodiments, the emulsifier component is present in an amount of about 3% to about 5% by weight of the emulsion.

In some embodiments, the emulsifier component is present in an amount of about 4% to about 7% by weight of the emulsion.

In some embodiments, the emulsion comprises an emulsifier component and a stiffening agent component, wherein the combined amount of emulsifier component and stiffening agent component is at least about 8% by weight of the emulsion.

In some embodiments, the emulsifier component comprises one or more substances independently selected from glyceryl fatty esters and sorbitan fatty esters.

In some embodiments, the emulsifier component comprises one or more substances independently selected from glyceryl stearate, and polysorbate 20.

In some embodiments, the emulsion further comprises a stabilizing agent component.

In some embodiments, the stabilizing agent component is present in an amount of about to about 5% by weight of the emulsion.

In some embodiments, the stabilizing agent component is present in an amount of about 0.1% to about 2% by weight of the emulsion.

In some embodiments, the stabilizing agent component is present in an amount of about to about 0.5% by weight of the emulsion.

In some embodiments, the stabilizing agent component comprises one or more substances independently selected from polysaccharides.

In some embodiments, the stabilizing agent component comprises xanthan gum.

In some embodiments, the emulsion further comprises a solvent component.

In some embodiments, the solvent component is present in an amount of about 10% to about 35% by weight of the emulsion.

In some embodiments, the solvent component is present in an amount of about 15% to about 30% by weight of the emulsion.

In some embodiments, the solvent component is present in an amount of about 20% to about 25% by weight of the emulsion.

In some embodiments, the solvent component comprises one or more substances independently selected from alkylene glycols and polyalkylene glycols.

In some embodiments, the solvent component comprises one or more substances independently selected from propylene glycol and polyethylene glycol.

In some embodiments, the emulsion comprises:
from about 35% to about 65% of water by weight of the emulsion;
from about 10% to about 40% of an oil component by weight of the emulsion;
from about 1% to about 9% of an emulsifier component by weight of the emulsion;
from about 10% to about 35% of a solvent component by weight of the emulsion;
from about 0.05% to about 5% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 35% to about 65% of water by weight of the emulsion;
from about 10% to about 24% of an oil component by weight of the emulsion;
from about 1% to about 9% of an emulsifier component by weight of the emulsion;
from about 10% to about 35% of a solvent component by weight of the emulsion;
from about 0.05% to about 5% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 40% to about 60% of water by weight of the emulsion;
from about 15% to about 30% of an oil component by weight of the emulsion;
from about 2% to about 6% of an emulsifier component by weight of the emulsion;
from about 15% to about 30% of a solvent component by weight of the emulsion;
from about 0.1% to about 2% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 40% to about 60% of water by weight of the emulsion;
from about 15% to about 30% of an oil component by weight of the emulsion;
from about 2% to about 6% of an emulsifier component by weight of the emulsion;
from about 15% to about 24% of a solvent component by weight of the emulsion;
from about 0.1% to about 2% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 15% to about 24% of an oil component by weight of the emulsion;
from about 3% to about 5% of an emulsifier component by weight of the emulsion;
from about 20% to about 25% of a solvent component by weight of the emulsion;
from about 0.3% to about 0.5% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 15% to about 24% of an oil component by weight of the emulsion;
from about 4% to about 7% of an emulsifier component by weight of the emulsion;
from about 20% to about 25% of a solvent component by weight of the emulsion;
from about 0.3% to about 0.5% of a stabilizing agent component by weight of the emulsion; and
from 0.5% to 1.5% of ruxolitinib, or a pharmaceutically acceptable salt thereof, by weight of the emulsion on a free base basis.

In some embodiments:
the oil component comprises one or more substances independently selected from petrolatums, fatty alcohols, mineral oils, triglycerides, and dimethicones;
the emulsifier component comprises one or more substances independently selected from glyceryl fatty esters and sorbitan fatty esters;
the solvent component comprises one or more substances independently selected from alkylene glycols and polyalkylene glycols; and
the stabilizing agent component comprises one or more substances independently selected from polysaccharides.

In some embodiments:
the oil component comprises one or more substances independently selected from white petrolatum, cetyl alcohol, stearyl alcohol, light mineral oil, medium chain triglycerides, and dimethicone;
the emulsifier component comprises one or more substances independently selected from glyceryl stearate and polysorbate 20;
the solvent component comprises one or more substances independently selected from propylene glycol and polyethylene glycol; and
the stabilizing agent component comprises xanthan gum.

In some embodiments, the emulsion comprises:
from about 35% to about 65% of water by weight of the emulsion;
from about 2% to about 15% of an occlusive agent component by weight of the emulsion;
from about 2% to about 8% of a stiffening agent component by weight of the emulsion;
from about 5% to about 15% of an emollient component by weight of the emulsion;
from about 1% to about 9% of an emulsifier component by weight of the emulsion;
from about 0.05% to about 5% of a stabilizing agent component by weight of the emulsion;
from about 10% to about 35% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 40% to about 60% of water by weight of the emulsion;
from about 5% to about 10% of an occlusive agent component by weight of the emulsion;
from about 2% to about 8% of a stiffening agent component by weight of the emulsion;
from about 7% to about 12% of an emollient component by weight of the emulsion;
from about 2% to about 6% of an emulsifier component by weight of the emulsion;
from about 0.1% to about 2% of a stabilizing agent by weight of the emulsion;
from about 15% to about 30% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 5% to about 10% of an occlusive agent component by weight of the emulsion;
from about 3% to about 6% of a stiffening agent component by weight of the emulsion;
from about 7% to about 13% of an emollient component by weight of the emulsion;
from about 3% to about 5% of an emulsifier component by weight of the emulsion;
from about 0.3% to about 0.5% of a stabilizing agent component by weight of the emulsion;
from about 20% to about 25% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 5% to about 10% of an occlusive agent component by weight of the emulsion;
from about 4% to about 7% of a stiffening agent component by weight of the emulsion;
from about 7% to about 13% of an emollient component by weight of the emulsion;
from about 4% to about 7% of an emulsifier component by weight of the emulsion;
from about 0.3% to about 0.5% of a stabilizing agent component by weight of the emulsion;
from about 20% to about 25% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
about 7% of an occlusive agent component by weight of the emulsion;
from about 4.5% to about 5% of a stiffening agent component by weight of the emulsion;
about 10% of an emollient component by weight of the emulsion;
from about 4% to about 4.5% of an emulsifier component by weight of the emulsion;
about 0.4% of a stabilizing agent component by weight of the emulsion;
about 22% of a solvent component by weight of the emulsion; and
from 0.5% to 1.5% of ruxolitinib, or a pharmaceutically acceptable salt thereof, by weight of the emulsion on a free base basis.

In some embodiments, the ruxolitinib, or pharmaceutically acceptable salt thereof, is present as ruxolitinib phosphate.

In some embodiments, the emulsion comprises 1.5% of ruxolitinib, or a pharmaceutically acceptable salt thereof, by weight of the emulsion.

In some embodiments, the emulsion comprises 1.5% of ruxolitinib phosphate by weight of the emulsion.

In some embodiments, the emulsion comprises 0.75% of ruxolitinib, or a pharmaceutically acceptable salt thereof, by weight of the emulsion.

In some embodiments, the emulsion comprises 0.75% of ruxolitinib phosphate by weight of the emulsion.

In some embodiments, the combined amount of the stiffening agent component and the emulsifier component is at least about 8% by weight of the emulsion.

In some embodiments:
the occlusive agent component comprises a petrolatum;
the stiffening agent component comprises one or more substances independently selected from one or more fatty alcohols;
the emollient component comprises one or more substances independently selected from mineral oils and triglycerides;
the emulsifier component comprises one or more substances independently selected from glyceryl fatty esters and sorbitan fatty esters;
the stabilizing agent component comprises one or more substances independently selected from polysaccharides; and
the solvent component comprises one or more substances independently selected from alkylene glycols and polyalkylene glycols.

In some embodiments:
the occlusive agent component comprises white petrolatum;
the stiffening agent component comprises one or more substances independently selected from cetyl alcohol and stearyl alcohol;
the emollient component comprises one or more substances independently selected from light mineral oil, medium chain triglycerides, and dimethicone;
the emulsifier component comprises one or more substances independently selected from glyceryl stearate and polysorbate 20;
the stabilizing agent component comprises xanthan gum; and
the solvent component comprises one or more substances independently selected from propylene glycol and polyethylene glycol.

In some embodiments, the emulsion further comprises an antimicrobial preservative component.

In some embodiments, the antimicrobial preservative component is present in an amount of about 0.05% to about 3% by weight of the emulsion.

In some embodiments, the antimicrobial preservative component is present in an amount of about 0.1% to about 1% by weight of the emulsion.

In some embodiments, the antimicrobial preservative component comprises one or more substances independently selected from alkyl parabens and phenoxyethanol.

In some embodiments, the antimicrobial preservative component comprises one or more substances independently selected from methyl paraben, propyl paraben, and phenoxyethanol.

In some embodiments, the emulsion further comprises a chelating agent component.

In some embodiments, the chelating agent component comprises edetate disodium.

As used herein, the term “emulsifier component” refers, in one aspect, to a substance, or mixtures of substances that maintains an element or particle in suspension within a fluid medium. In some embodiments, the emulsifier component allows an oil phase to form an emulsion when combined with water. In some embodiments, the emulsifier component refers to one or more non-ionic surfactants.

As used herein, the term “occlusive agent component” refers to a hydrophobic agent or mixtures of hydrophobic agents that form an occlusive film on skin that reduces transepidermal water loss (TEWL) by preventing evaporation of water from the stratum corneum.

As used herein, the term “stiffening agent component” refers to a substance or mixture of substances that increases the viscosity and/or consistency of the cream or improves the rheology of the cream.

As used herein, the term “emollient component” refers to an agent that softens or soothes the skin or soothes an irritated internal surface.

As used herein, the term “stabilizing agent component” refers to a substance or mixture of substances that improves the stability of the cream and/or the compatibility of the components in the cram. In some embodiments, the stabilizing agent component prevents agglomeration of the emulsion and stabilizes the droplets in the oil-in-water emulsion.

As used herein, the term “solvent component” is a liquid substance or mixture of liquid substances capable of dissolving ruxolitinib (or its salt) or other substances in the cream. In some embodiments, the solvent component is a liquid substance or mixture of liquid substances in which ruxolitinib, or its pharmaceutically acceptable salt, has reasonable solubility. For example, solubilities of ruxolitinib (free base) or its phosphate salt (1:1 salt) are reported in Table 1. In some embodiments, a solvent is a substance or mixture thereof, in which ruxolitinib, or its pharmaceutically acceptable salt (whichever is used), has a solubility of at least about 10 mg/mL or greater, at least about 15 mg/mL or greater, or at least about 20 mg/mL or greater, when measured as described in Example 2.

As used herein, the phrase “antimicrobial preservative component” is a substance or mixtures of substances which inhibits microbial growth in the cream.

As used herein, the phrase “chelating agent component” refers to a compound or mixtures of compounds that has the ability to bind strongly with metal ions.

As used herein, “% by weight of the emulsion” means the percent concentration of the component in the emulsion is on weight/weight basis. For example, 1% w/w of component A=[(mass of component A)/(total mass of the emulsion)]×100.

As used herein, “% by weight of the emulsion on a free base basis” of ruxolitinib, or pharmaceutically acceptable salt thereof” means that the % w/w is calculated based on the weight of ruxolitinib in the total emulsion. For example, “1.5% w/w on a free base basis” of ruxolitinib phosphate means that for 100 grams of total formulation, there are 1.98 grams of ruxolitinib phosphate in the emulsion (which equates to 1.5 grams of the free base, ruxolitinib).

As used herein, the term “component” can mean one substance or a mixture of substances.

As used herein, the term “fatty acid” refers to an aliphatic acid that is saturated or unsaturated. In some embodiments, the fatty acid is in a mixture of different fatty acids. In some embodiments, the fatty acid has between about eight to about thirty carbons on average. In some embodiments, the fatty acid has about 12 to 20, 14-20, or 16-18 carbons on average. Suitable fatty acids include, but are not limited to, cetyl acid, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, behenic acid, isobehenic acid, and arachidonic acid, or mixtures thereof.

As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12 to about 20, about 14 to about 20, or about 16 to about 18 carbons on average. Suitable fatty alcohols include, but are not limited to, stearyl alcohol, lauryl alcohol, palmityl alcohol, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “polyalkylene glycol”, employed alone or in combination with other terms, refers to a polymer containing oxyalkylene monomer units, or copolymer of different oxyalkylene monomer units, wherein the alkylene group has 2 to 6, 2 to 4, or 2 to 3 carbon atoms. As used herein, the term “oxyalkylene”, employed alone or in combination with other terms, refers to a group of formula —O-alkylene-. In some embodiments, the polyalkylene glycol is polyethylene glycol.

As used herein, the term, “sorbitan fatty ester” includes products derived from sorbitan or sorbitol and fatty acids and, optionally, poly(ethylene glycol) units, including sorbitan esters and polyethoxylated sorbitan esters. In some embodiments, the sorbitan fatty ester is a polyethoxylated sorbitan ester.

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), 5th ed., which is incorporated herein by reference in its entirety.

As used herein, the term “polyethoxylated sorbitan ester” refers to a compound, or mixture thereof, derived from the ethoxylation of a sorbitan ester. The polyoxethylene portion of the compound can be between the fatty ester and the sorbitan moiety. As used herein, the term “sorbitan ester” refers to a compound, or mixture of compounds, derived from the esterification of sorbitol and at least one fatty acid. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE(20) sorbitan monolaurate), 21 (POE(4) sorbitan monolaurate), 40 (POE(20) sorbitan monopalmitate), 60 (POE(20) sorbitan monostearate), 60K (POE(20) sorbitan monostearate), 61 (POE(4) sorbitan monostearate), 65 (POE(20) sorbitan tristearate), 80 (POE(20) sorbitan monooleate), 80K (POE(20) sorbitan monooleate), 81 (POE(5) sorbitan monooleate), and 85 (POE(20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is incorporated herein by reference in its entirety. In some embodiments, the polyethoxylated sorbitan ester is a polysorbate. In some embodiments, the polyethoxylated sorbitan ester is polysorbate 20.

As used herein, the term “glyceryl fatty esters” refers to mono-, di- or triglycerides of fatty acids. The glyceryl fatty esters may be optionally substituted with sulfonic acid groups, or pharmaceutically acceptable salts thereof. Suitable fatty acids for deriving glycerides of fatty acids include, but are not limited to, those described herein. In some embodiments, the glyceryl fatty ester is a mono-glyceride of a fatty acid having 12 to 18 carbon atoms. In some embodiments, the glyceryl fatty ester is glyceryl stearate.

As used herein, the term “triglycerides” refers to a triglyceride of a fatty acid. In some embodiments, the triglyceride is medium chain triglycerides.

As used herein, the term “alkylene glycol” refers to a group of formula —O-alkylene-, wherein the alkylene group has 2 to 6, 2 to 4, or 2 to 3 carbon atoms. In some embodiments, the alkylene glycol is propylene glycol (1,2-propanediol).

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present disclosure can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer.

### Kits

The present invention also includes pharmaceutical kits useful, for example, in the treatment and/or prevention of prurigo nodularis, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to be JAK inhibitors according to at least one assay described herein.

### Example 1. Upregulated JAK-STAT Pathway Expression

RNA are isolated from formalin-fixed paraffin embedded (FFPE) skin biopsies from active disease, untreated, prurigo nodularis patients. RNA are processed using the nCounter autoimmune profiling codeset (770 genes) or the neuropathology profiling codeset (770 genes) (Nanostring, USA), according to the manufacturer's protocol. After an 18 h hybridization, the samples were run on an nCounter SPRINT Profiler (Nanostring, USA). Data was analyzed using nSolver 4.0 Advanced Analysis software (Nanostring, USA). P-values were adjusted using the Benjamini-Yekutieli false discovery rate method.

### Ruxolitinib-Mediated Pharmacological Inhibition of PN Pathophysiology

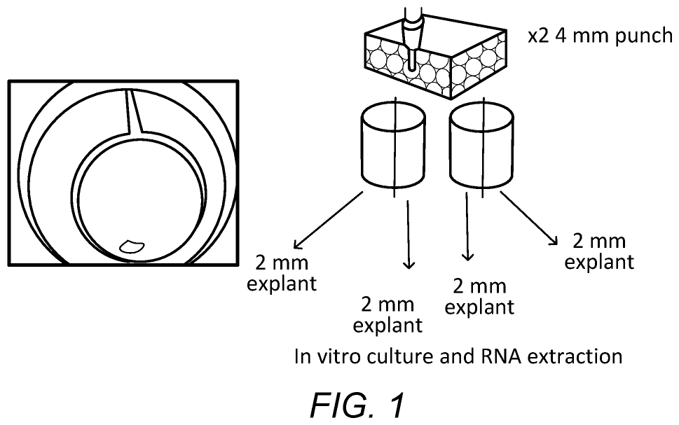

Full-thickness cutaneous skin biopsies were obtained from active disease, untreated, prurigo nodularis patients. Single 4 mm-punch biopsies were obtained from the same lesion for each patient and longitudinally divided into two pieces (see FIG. 1). FIG. 1 depicts a representation of skin punch biopsies used for the JAK1/JAK2 mediated pharmacological inhibition of PN pathophysiology. Biopsies were cultured for 8 days in KBM media+CaCl₂ and refreshed every 2-3 days. Ruxolitinib phosphate or DMSO (control) was added to the cell culture media. Conditioned supernatants collected during/prior to media refresh were stored at −for subsequent analysis. On day 8, cultures were terminated and tissue RNA isolated for subsequent analysis.

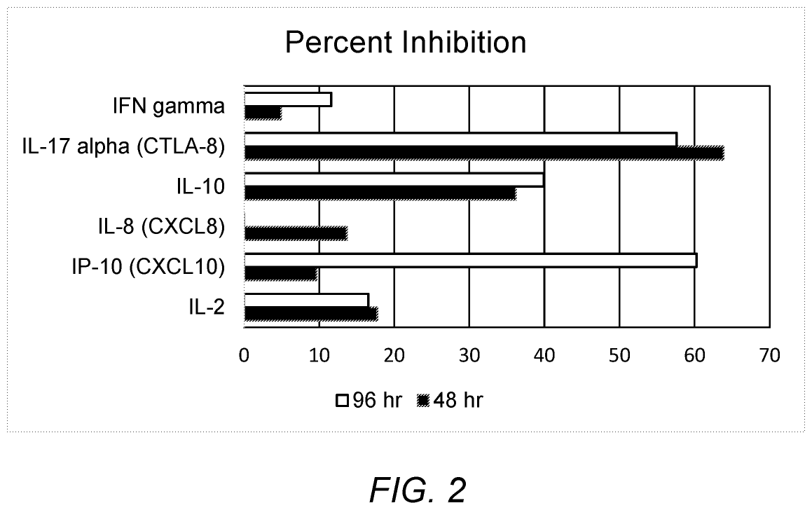

Collected supernatants were analyzed to quantify chemokines, cytokines, and growth factors secreted by skin explants in culture and which are involved in inflammation. A total of 51 proteins were detected and quantified in culture supernatants by Procarta Multiplex Immunoassay (Thermo Fisher, Waltham, MA). Supernatants and standards were incubated at 4° C. overnight. Assay plates were read on a Luminex 200 Instrument (Luminex Corporation, Austin, TX). The concentration was extrapolated from the antigen standard curve of each analyte. The percent inhibition determined for each analyte as the difference in protein concentration in cultures with DMSO (C_{D}) versus those with ruxolitinib (C_{J}), (C_{D}−C_{J})/C_{D} (see FIG. 2).

### Results

Ruxolitinib was shown to inhibit key chemokines, cytokines, and growth factors involved in key disease pathways (see Table 1 and FIG. 2). Further, when the same assays were conducted for a JAK1 inhibitor, 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, IL-5 and IL-22 were also shown to be inhibited at 48 hours (percent inhibition of 20.25% and 11.16%, respectively. These key chemokines, cytokines, and growth factors are involved in inflammation, pruritus (itching), or lesion development that can be associated with PN. For example, PN lesional skin has been shown to have increased levels of CXCL8, CXCL10, and interferon-gamma when compared to non-lesional control skin (Tsoi et al., J Allergy Clin Immunol 149, 1329-39 (2022)).

Further other cytokines, such as IL-31 and IL-17, have also been shown to play a role in PN. For example, CD4+ T-cells has been shown to significantly infiltrate lesional PN skin as compared to non-lesional control skin (Wong, et al., J Investigative Dermatology, 140(3), 702-706.e2 (2020)). CD4+ T-cells can express interleukin 31(IL-31), which has been shown to be a mediator of pruritus—an important symptom of PN (Stander, et al., N Engl J Med, 382:706-716 (2020)). The expressed IL-31 has also been shown to play a role in the development of PN with inhibition of IL-31 by nemolizumab resulting in improvement in pruritus and skin lesions in PN patients (Stander, supra). When PN skin was cultured nemolizumab (an IL-31 inhibitor), a reduction in IL-17 levels in lesional skin was observed, suggesting that inhibiting IL31R signaling can impact IL-17 expression (Tsoi, supra). Further, cells expressing IL-17 has been shown to be increased in the dermis of PN lesional skin as compared to control skin (Wong, supra). Activated T-cells have also shown increased IL-22 cytokine expression in patients with PN compared with healthy controls (Belzberg, et al., J Invest Dermatol, 141(9):2208-2218.e14 (2021)). Further, IL-2 is critical to the development, maintenance and function of the infiltrating CD4+ T-cells (Furtado, J Exp Med, 196(6):851-7 (2002)) and is a strong itch mediator (Xie, et al., J Dermatol, 46(3), 177-185 2019)). Further, the epidermis of PN skin has been shown to be positive for STATE, a marker for Th2 cytokines, such as IL-5 and IL-31 (Mullins, et al., NLM, StatPearls Publishing, https://www.ncbi.nlm.nih.gov/books/NBK459204), Sep. 14, 2021)). Finally, keratinocytes are implicated in the pathogenesis of PN, particularly around pruritus (Zhong et al, Acta Dermato-Venereologicavolume 99, 579-5861 May (2019)). In skin, keratinocytes produce IL-34.

Hence, ruxolitinib has been shown to inhibit multiple key chemokines, cytokines, and growth factors involved in PN and/or the underlying inflammation and pruritus associated with PN.

TABLE A
(showing percent inhibition due to ruxolitinib)

| Analyte | 48 hr | 96 hr |
|---|---|---|
| IP-10 (CXCL10) | 9.7 | 60.3 |
| IL-8 (CXCL8) | 13.7 | ** |
| IL-10 | 36.2 | 39.9 |
| IL-17 alpha (CTLA-8) | 63.9 | 57.6 |
| IFN-gamma | 5.0 | 11.6 |
| IL-2 | 17.8 | 16.6 |

** no measureable inhibition

### Example 2: Preparation of Oil-In-Water Cream Formulations of Ruxolitinib Phosphate

First, in order to determine the solubility of ruxolitinib (free base) or its 1:1 phosphate salt, approximately 5 mL of a potential solvent was added to approximately 50 mg of the API or its salt at room temperature. The mixtures were suspended and rotated on a wheel. If the mixtures became clear solutions, more solid material was added. The suspensions were then suspended over 24 hours. The samples were filtered through 0.2 micron filters. The liquid portions were collected and diluted with 50/50 water methanol/water. The concentrations of the diluted samples were analyzed by HPLC. When the free base or salt was fairly insoluble, the results are approximate only.

TABLE 1

| | Solubility of Phosphate | Solubility of Free |
|---|---|---|
| Potential Solvent | Salt (mg/mL) | Base (mg/mL) |
| Water | 2.7 | 2.0 |
| pH 4, citric buffer, 0.1M | 1.5 | 1.1 |
| pH 6, citric buffer, 0.1M | 0.2 | 0.15 |
| Ethanol | 7.3 | 5.5 |
| Isopropanol | 0.6 | 0.45 |
| Benzyl alcohol | 3 | 2.3 |
| Propylene glycol | 24 | 18.2 |
| PEG 200 | 23 | 17.4 |
| PEG 300 | 14 | 10.6 |
| Glycerin | 11 | 8.3 |
| Transcutol | 10 | 7.6 |
| Trolamine | 51 | 38.6 |
| Water/PEG 200 (50/50) | 23 | 17.4 |
| Water/glyercin (50/50) | 21 | 15.9 |
| Water/glycerin/trolamine | 18 | 13.6 |
| (40/40/20) | | |
| Isopropyl myristate | <0.1 | 0.08 |
| Isosorbide dimethyl ether | 0.4 | 0.3 |
| Mineral oil | <0.1 | 0.08 |
| Olelyl alcohol | 0.1 | 0.08 |
| Dimethicone | <0.2 | 0.15 |
| C₁₂₋₁₅ alcohol benzoate | <0.2 | 0.15 |
| Caprylic triglyceride | <0.2 | 0.15 |

An oil-in-water cream formulation was prepared for 1:1 ruxolitinib phosphoric acid salt at 0.5, 1.0 and 1.5% by weight of the formulation (free base equivalent). The compositions for a gram tube are provided in Table 2 below. The formulation for three strengths were identical except for adjustments to the purified water quantity based on the amount of active ingredient. All excipients used in the formulation were compendial grade (i.e., USP/NF or BP) or are approved for use in topical products.

The quantitative formulae for representative 400 kg batches of the cream formulation at 1.0 and 1.5% are also provided in Tables 3, 4, and 5, respectively.

The oil-in-water cream formulations were synthesized according to the following procedure at either a 3.5 kg or 400 kg scale (when made at a 3.5 kg batch size, the amounts in Tables 3-5 were scaled appropriately). Some batches were subject to minor changes associated with scale-up, such as the size of mixing vessels and mixers. Generally, overhead mixer with high and low shear mixing blades are suitable for the process.

### Procedure

1. A paraben phase was prepared by mixing methyl and propyl parabens with a portion of the propylene glycol (see % in Tables 2-5).
2. Next, a xanthan gum phase was prepared by mixing xanthan gum with propylene glycol (see % in Table 2-5).
3. An oil phase was then prepared by mixing light mineral oil, glyceryl stearate, polysorbate 20, white petrolatum, cetyl alcohol, stearyl alcohol, dimethicone and medium chain triglycerides. The phase is heated to 70-80° C. to melt and form a uniform mixture.
4. The aqueous phase was next prepared by mixing purified water, polyethylene glycol, and disodium EDTA. The phase is heated to 70-80° C.
5. The aqueous phase of step 4, paraben phase of step 1, and Example 2 (phosphate salt of API) were combined to form a mixture.
6. The xanthan gum phase from step 2 was then added to the mixture from step 5.
7. The oil phase from step 3 was then combined under high shear mixing with the mixture from step 6 to form an emulsion.
8. Phenoxyethanol was then added to the emulsion from step 7. Mixing was continued, and then the product was cooled under low shear mixing.

TABLE 2

| | | | Percentage | |
|---|---|---|---|---|
| FORMULA | | | of Total | Grams/ |
| PHASE | COMPONENT | Function | (% w/w) | Tube |
| Paraben | Propylene Glycol | Solvent | 10.00 | 1.5 |
| | USP | | | |
| | Methyl Paraben | Antimicrobial | 0.10 | 0.015 |
| | NF | preservative | | |
| | Propyl Paraben | Antimicrobial | 0.05 | 0.0075 |
| | NF | preservative | | |
| Xanthan | Propylene Glycol | Solvent | 5.00 | 0.75 |
| Gum | USP | | | |
| | Xanthan Gum NF | Suspending, | 0.40 | 0.06 |
| | | stabilizing, | | |
| | | viscosity- | | |
| | | increasing agent | | |
| Oil | Light Mineral Oil | Emollient, solvent | 4.00 | 0.6 |
| | NF | | | |
| | Glyceryl Stearate | Emulsifier | 3.00 | 0.45 |
| | SE | | | |
| | Polysorbate 20 | Emulsifying/ | 1.25 | 0.1875 |
| | NF | stabilizing agent | | |
| | White Petrolatum | Occlusive agent | 7.00 | 1.05 |
| | USP | | | |
| | Cetyl Alcohol NF | Stiffening agent, | 3.00 | 0.45 |
| | | consistency | | |
| | | improver | | |
| | Stearyl Alcohol | Stiffening agent | 1.75 | 0.2625 |
| | NF | | | |
| | Dimethicone 360 | Skin protectant | 1.00 | 0.15 |
| | NF | | | |
| | Medium Chain | Emollient, solvent | 5.00 | 0.75 |
| | Triglyceride NF | | | |
| Aqueous/ | Purified Water | Solvent | 50.24- | 7.536- |
| Active | USP | | 48.92 | 7.338 |
| | Edetate Disodium | Chelating agent | 0.05 | 0.0075 |
| | USP | | | |
| | Polyethylene | Solvent | 7.00 | 1.05 |
| | Glycol USP | | | |
| | Example 2 * | Active | 0.66- | 0.099- |
| | | | 1.98 | 0.297 |
| Final | Phenoxyethanol | Antimicrobial | 0.50 | 0.075 |
| | BP | preservative | | |
| | Total | | 100.00% | 15 |

TABLE 3

| Ingredient | Kilograms | Percentage (w/w) |
|---|---|---|
| Ruxolitinib phosphate | 2.64 (phosphate | 0.66 (phosphate s |
| | salt)/ | alt)/ |
| | 2.0 (free base) | 0.5 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 201 | 50.25 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

TABLE 4

| Ingredient | Kilograms | Percentage (w/w) |
|---|---|---|
| Ruxolitinib phosphate | 5.28 (phosphate | 1.32 (phosphate |
| | salt)/ | salt)/ |
| | 4.0 (free base) | 1.00 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 198.5 | 49.6 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

TABLE 5

| Ingredient | Kilograms | Percentage (w/w) |
|---|---|---|
| Ruxolitinib phosphate | 7.92 (phosphate | 1.98 (phophate |
| | salt)/ | salt)/ |
| | 6.0 (free base) | 1.5 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 195.5 | 48.9 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

The batches were tested for stability at 25° C. and found to be stable up to 24 months with a pH consistent with the pH range described supra (see Table 7, 9, 11, 12, 13, 15, 17, and 19 in U.S. Patent Publ. No. 2015/0250790, which is incorporated herein by reference in its entirety).

Various modifications of the invention, in addition to those described herein, will be apparent to those skilled in the art from the foregoing description. Such modifications are also intended to fall within the scope of the appended claims. U.S. Provisional Application No. 63/183,249, filed May 3, 2021, and its U.S. non-provisional application entitled “JAK1 Pathway Inhibitors for Treatment Of Prurigo Nodularis”, filed May 3, 2022, are incorporated herein by reference in their entireties. Each reference, including all patent, patent applications, and publications, cited in the present application is incorporated herein by reference its entirety.

What is claimed is:

1. A method for treating prurigo nodularis in a subject, said method comprising administering to the subject a therapeutically effective amount of ruxolitinib, or a pharmaceutically acceptable salt thereof.

2. The method of claim 1, wherein the ruxolitinib, or a pharmaceutically acceptable salt thereof, is ruxolitinib phosphate.

3. The method of claim 1, wherein the ruxolitinib, or a pharmaceutically acceptable salt thereof, is administered topically to an affected skin area.

4. The method of claim 1, wherein the ruxolitinib, or a pharmaceutically acceptable salt thereof, is an administered as a topical formulation.

5. The method of claim 4, wherein the topical formulation is an administered as a topical formulation comprising about 0.1% to about 3% of the ruxolitinib, or the pharmaceutically acceptable salt thereof, on a free base basis.

6. The method of claim 4, wherein the topical formulation is an administered as a topical formulation comprising about 0.5% to about 1.5% of the ruxolitinib, or the pharmaceutically acceptable salt thereof, on a free base basis.

7. The method of claim 4, wherein the topical formulation is an administered as a topical formulation comprising about 0.75% to about 1.5% of the ruxolitinib, or the pharmaceutically acceptable salt thereof, on a free base basis.

8. The method of claim 4, wherein the topical formulation is an administered as a topical formulation comprising about 0.75% of the ruxolitinib, or the pharmaceutically acceptable salt thereof, on a free base basis.

9. The method of claim 4, wherein the topical formulation is an administered as a topical formulation comprising about 1% of the ruxolitinib, or the pharmaceutically acceptable salt thereof, on a free base basis.

10. The method of claim 4, wherein the topical formulation is an administered as a topical formulation comprising about 1.5% of the ruxolitinib, or the pharmaceutically acceptable salt thereof, on a free base basis.

11. The method of claim 4, wherein the topical formulation is administered twice per day (BID).

12. The method of claim 4, wherein the topical formulation is administered once per day (QD).

13. The method of claim 4, wherein the topical formulation is administered for at least 12 weeks.

14. The method of claim 4, wherein the topical formulation is a cream formulation.

15. The method of claim 1, wherein the subject achieves an IGA score of 0 or 1 with ≥2 grade improvement from baseline.

16. The method of claim 1, wherein the subject achieves an improvement in a subject's response to Itch NRS from baseline.

17. The method of claim 1, wherein the subject achieves about a 5%, about a 10%, about a 20%, about a 30%, about a 40%, about a 50%, about a 60%, about a 70%, about a 80%, about a 90%, or about a 95% improvement in a subject's response to Itch NRS from baseline.

18. The method of claim 1, wherein the subject achieves about a 5%, about a 10%, about a 20%, about a 30%, about a 40%, about a 50%, about a 60%, about a 70%, about a 80%, about a 90%, or about a 95% improvement in a subject's response in Prurigo Activity Score (PAS) from baseline.

19. The method of claim 1, wherein the subject achieves about a 5%, about a 10%, about a 20%, about a 30%, about a 40%, about a 50%, about a 60%, about a 70%, about a 80%, about a 90%, or about a 95% improvement in a subject's response in Itchy Quality of Life (Itchy QoL) from baseline.

20. The method of claim 1, wherein the subject achieves about a 5%, about a 10%, about a 20%, about a 30%, about a 40%, about a 50%, about a 60%, about a 70%, about a 80%, about a 90%, or about a 95% improvement in a subject's response in Dynamic Pruritus Score (DPS) from baseline.

21. The method of claim 1, wherein the subject achieves about a 5%, about a 10%, about a 20%, about a 30%, about a 40%, about a 50%, about a 60%, about a 70%, about a 80%, about a 90%, or about a 95% improvement in a subject's response to DLQI from baseline.

22. The method of claim 1, wherein the subject has an itch Numerical Rating Scale of at least 4 at baseline.

23. The method of claim 1, wherein the subject is aged 18 or older.

24. The method of claim 1, wherein the ruxolitinib, or pharmaceutically acceptable salt thereof, is administered in combination with a further therapeutic agent.
